(19) United States
(12) Patent Application Publication
(10) Pub. No.: US 2023/0193364 A1
(43) Pub. Date: Jun. 22, 2023

(54) A GENETIC TEST TO PREDICT ANTI-TNF DRUG RESPONSE

(71) Applicant: University of Ulster, Londonderry (GB)

(72) Inventors: David Gibson, Londonderry (GB); Tony Bjourson, Londonderry (GB); Cathy McGough, Londonderry (GB); Martin Crockard, Crumlin (GB); John Lamont, Crumlin (GB)

(73) Assignee: University of Ulster, Londonderry (GB)

(21) Appl. No.: 17/999,616

(22) PCT Filed: May 21, 2021
(86) PCT No.: PCT/EP2021/063722

§ 371 Date: Nov. 22, 2022

(30) Foreign Application Priority Data
May 22, 2020 (GB) 2007698.0

(51) Int. Cl. C12Q 1/6827; C12Q 1/6876

(52) U.S. Cl. CPC C12Q 1/6827; C12Q 1/6876; C12Q 2600/106; C12Q 2600/158

(57) ABSTRACT

This invention relates to predicting a subjects responsiveness to biologic therapy of rheumatoid arthritis. The invention provides a method for predicting responsiveness to anti-tumour necrosis factor therapy of rheumatoid arthritis in a subject, the method comprising the steps of: (a) Providing a biological sample; (b) detecting the presence, absence, or quantitative level of a first marker or an expression product thereof, wherein the first marker is at the HLA DRB1 gene; (c) detecting the presence, absence, or quantitative level of a second marker, or an expression product thereof, wherein the second marker is at the CD226 gene; (d) correlating the presence, absence, or quantitative level of the first marker and the presence, absence, or quantitative level of the second marker; to the predicted responsiveness to anti-tumour necrosis factor therapy of rheumatoid arthritis in the subject.

### FIELD OF THE INVENTION

The present invention relates to predicting a subject's responsiveness to biologic therapy of rheumatoid arthritis.

### BACKGROUND OF THE INVENTION

Anti-tumour necrosis factor alpha (TNF) blocking agents are effective at reducing disease activity measures in approximately 60-70% of rheumatoid arthritis (RA) patients. As TNF is a key driver of joint inflammation, in those who respond, anti-TNF biologic drugs reduce immune cell infiltration into the joint and diminish joint destruction. In the absence of a widely accepted test which can predict how patients will respond prior to prescribing, commonly used biologic treatment strategies currently exceed the cost effectiveness thresholds set by the National Institute for Health and Care Excellence (NICE). Many studies have assessed whether various clinical, proteomic and genetic characteristics could act as reliable predictive factors to stratify future responders with mixed success, but few promising biomarkers have been independently validated.

Lower baseline health assessment questionnaire scores and concurrent use of methotrexate have been found to associate with a good response to anti-TNF treatment by the European League Against Rheumatism (EULAR) criteria. Normal body mass index, lower baseline disease activity and non-smoker status have also been associated with improved rates of response to anti-TNF. The association of rheumatoid factor and anti-citrullinated protein antibodies (ACPA) with response to TNF-inhibitors has been investigated in several studies with conflicting findings. A recent pilot study demonstrated how clinical factors can be included with baseline serum myeloid-related protein (MRP) 8/14 to design a treatment algorithm capable of predicting anti-TNF response.

Many single nucleotide polymorphism studies have demonstrated associations with anti-TNF therapy treatment response. Genetic variants of components which map to T cell function have been found to associate with response in RA patients, including interleukin-1 receptor-associated kinase 3 (IRAK3), which negatively regulates toll-like receptor (TLR) signalling, and conserved helix-loop-helix ubiquitous kinase (CHUK) and myeloid differentiation primary response protein (MyD88), which activate or inhibit nuclear factor-κB (NF-κB) signalling. MyD88 and CHUK were previously associated with etanercept response. A contradictory study found no response association with genetic variants of CD226, AF3/FMR2 family, member 3 (AFF3) in addition to CHUK and MyD88. Genetic variants of the mitogen-activated protein kinase (MAPK) signalling pathway components also showed associations with infliximab and adalimumab responders in a study of RA patients.

Biologic drugs have revolutionised the treatment of Rheumatoid Arthritis (RA), however these therapies are expensive and exhibit a high non-response rate (30%).

Predicting response to anti-TNF drugs at baseline remains an elusive goal in RA management.

Therefore, there is a need for predictive tests which predict future responsiveness to biologic drugs in RA patients before initiating treatment. In particular, there is a need for predictive tests which predict future responsiveness to anti-TNF treatment in RA patients before initiating treatment.

### SUMMARY OF THE INVENTION

According a first aspect of the present invention, there is provided a method for predicting responsiveness to anti-tumour necrosis factor therapy in a subject, the method comprising the steps of:

b) detecting the presence, absence, or quantitative level of a first marker, or an expression product thereof;

d) correlating the presence, absence, or quantitative level of the first marker to the predicted responsiveness to anti-tumour necrosis factor therapy in the subject.

Optionally, the method comprises the steps of:

b) detecting the presence, absence, or quantitative level of a second marker, or an expression product thereof;

d) correlating the presence, absence, or quantitative level of the second marker to the predicted responsiveness to anti-tumour necrosis factor therapy in the subject.

Optionally, the method comprises the steps of:

b) detecting the presence, absence, or quantitative level of a first marker, or an expression product thereof;

c) detecting the presence, absence, or quantitative level of a second marker, or an expression product thereof;

d) correlating the presence, absence, or quantitative level of the first marker and the presence, absence, or quantitative level of the second marker; to the predicted responsiveness to anti-tumour necrosis factor therapy in the subject.

Optionally, the method comprises the steps of:

a) Providing a sample;

b) detecting the presence, absence, or quantitative level of a first marker, or an expression product thereof, in the sample;

c) detecting the presence, absence, or quantitative level of a second marker, or an expression product thereof, in the sample;

d) correlating the presence, absence, or quantitative level of the first marker and the presence, absence, or quantitative level of the second marker in the sample; to the predicted responsiveness to anti-tumour necrosis factor therapy in the subject.

Optionally, the anti-tumour necrosis factor therapy is for the treatment of a disorder in the subject.

Optionally, the predicted responsiveness to anti-tumour necrosis factor therapy in the subject is predicted responsiveness to anti-tumour necrosis factor therapy of the disorder in the subject.

Optionally, the disorder is selected from the group comprising: arthritis, osteoarthritis, rheumatoid arthritis, lupus, gout, gouty arthritis, infectious arthritis, psoriasis, and psoriatic arthritis.

Optionally, the disorder is an autoimmune disorder. Optionally, the disorder is an autoimmune disease.

Optionally, the anti-tumour necrosis factor therapy is for the treatment of an autoimmune disorder in the subject. Optionally, the autoimmune disorder is arthritis. Optionally, the autoimmune disorder is rheumatoid arthritis. Optionally, the autoimmune disorder comprises an autoimmune disorder selected from the group comprising: rheumatoid arthritis, lupus, celiac disease, diabetes mellitus type 1, Graves' disease, inflammatory bowel disease, multiple sclerosis, psoriasis, and systemic lupus erythematosus.

Optionally, the anti-tumour necrosis factor therapy is for the treatment of rheumatoid arthritis in the subject. Further optionally, the predicted responsiveness to anti-tumour necrosis factor therapy in the subject is predicted responsiveness to anti-tumour necrosis factor therapy of rheumatoid arthritis in the subject. Optionally, the anti-tumour necrosis factor therapy is for the treatment of rheumatoid arthritis in the subject, and the predicted responsiveness to anti-tumour necrosis factor therapy in the subject is predicted responsiveness to anti-tumour necrosis factor therapy of rheumatoid arthritis in the subject.

Optionally, there is provided a method for predicting responsiveness to anti-tumour necrosis factor therapy of rheumatoid arthritis in a subject, the method comprising the steps of:

b) detecting the presence, absence, or quantitative level of a first marker, or an expression product thereof;

d) correlating the presence, absence, or quantitative level of the first marker to the predicted responsiveness to anti-tumour necrosis factor therapy of rheumatoid arthritis in the subject.

Optionally, the method comprises the steps of:

b) detecting the presence, absence, or quantitative level of a second marker, or an expression product thereof;

d) correlating the presence, absence, or quantitative level of the second marker to the predicted responsiveness to anti-tumour necrosis factor therapy of rheumatoid arthritis in the subject.

Optionally, the method comprises the steps of:

b) detecting the presence, absence, or quantitative level of a first marker, or an expression product thereof;

c) detecting the presence, absence, or quantitative level of a second marker, or an expression product thereof;

d) correlating the presence, absence, or quantitative level of the first marker and the presence, absence, or quantitative level of the second marker; to the predicted responsiveness to anti-tumour necrosis factor therapy of rheumatoid arthritis in the subject.

Optionally, the method comprises the steps of:

a) Providing a sample;

b) detecting the presence, absence, or quantitative level of a first marker, or an expression product thereof, in the sample;

c) detecting the presence, absence, or quantitative level of a second marker, or an expression product thereof, in the sample;

d) correlating the presence, absence, or quantitative level of the first marker and the presence, absence, or quantitative level of the second marker in the sample; to the predicted responsiveness to anti-tumour necrosis factor therapy of rheumatoid arthritis in the subject.

Optionally, the sample comprises a biological sample. Optionally, the sample is a biological sample.

Optionally, the first marker is a first biomarker. Optionally, the second marker is a second biomarker.

Optionally, the sample substantially comprises a blood sample. Optionally, the sample comprises a blood sample. Optionally, the sample is a blood sample.

Optionally, the sample substantially comprises a tissue sample. Optionally, the sample comprises a tissue sample. Optionally, the sample is a tissue sample.

Optionally, the first marker is at the HLA gene complex.

Optionally, the first marker is at the HLA-DRB1 gene.

Optionally, the first marker comprises at least part of the nucleic acid sequence of the HLA DRB1*0404 allele.

Optionally, the first marker comprises at least part of the nucleic acid sequence of SEQ ID NO: 1.

Optionally, the first marker comprises at least part of a nucleic acid sequence selected from the nucleic acid sequence of the HLA DRB1*0404 allele, and the nucleic acid sequence of SEQ ID NO: 1.

Optionally, the second marker is at the CD226 gene.

Optionally, the second marker comprises at least part of the nucleic acid sequence of the rs763361 single nucleotide polymorphism.

Optionally, the second marker comprises at least part of the nucleic acid sequence of the rs763361 single nucleotide polymorphism at the CD226 gene. Optionally, the second marker comprises at least part of the nucleic acid sequence of the CD226 gene having the rs763361 single nucleotide polymorphism.

Optionally, the nucleic acid sequence of the rs763361 single nucleotide polymorphism is at least part of the sequence of the CD226 gene comprising the rs763361 single nucleotide polymorphism.

Optionally, the second marker comprises the nucleic acid sequence of the rs763361 single nucleotide polymorphism. Optionally, the second marker is the nucleic acid sequence of the rs763361 single nucleotide polymorphism.

Optionally, the second marker comprises at least part of a nucleic acid sequence selected from the group comprising the nucleic acid sequence of the rs763361 single nucleotide polymorphism, and the nucleic acid sequence of SEQ ID NO: 2.

Optionally, the second marker comprises at least part of the nucleic acid sequence of rs763361 cytosine single nucleotide polymorphism.

Optionally, the second marker comprises the nucleic acid sequence of the rs763361 cytosine single nucleotide polymorphism. Optionally, the second marker is the nucleic acid sequence of the rs763361 cytosine single nucleotide polymorphism.

Optionally, the second marker comprises at least part of a nucleic acid sequence selected from the group comprising the nucleic acid sequence of the rs763361 cytosine single nucleotide polymorphism, and the nucleic acid sequence of SEQ ID NO: 2.

Optionally, the second marker comprises at least part of the nucleic acid sequence of SEQ ID NO: 2.

Optionally, the second marker comprises the nucleic acid sequence of SEQ ID NO: 2. Optionally, the second marker is the nucleic acid sequence of SEQ ID NO: 2.

Optionally, absence of the first marker is homozygous absence of the first marker.

Optionally, presence of the first marker is homozygous presence of the first marker. Alternatively, presence of the first marker is heterozygous presence of the first marker.

Optionally, absence of the second marker is homozygous absence of the second marker.

Optionally, presence of the second marker is homozygous presence of the second marker. Alternatively, presence of the second marker is heterozygous presence of the second marker.

Optionally, the presence of the first marker indicates that the subject is predicted to be responsive to anti-tumour necrosis factor therapy of rheumatoid arthritis.

Optionally, the absence of the second marker indicates that the subject is predicted to be responsive to anti-tumour necrosis factor therapy of rheumatoid arthritis.

Optionally, the method further comprises the step of evaluating the DAS28 score of the subject.

Optionally, the subject has a DAS28 score of >5.1. Optionally, the subject has a DAS28 score of greater than 5.1. Optionally, the subject had a DAS28 score of >5.1 when originally assessed for anti-tumour necrosis factor therapy. Optionally, the subject had a DAS28 score of greater than 5.1 when originally assessed for anti-tumour necrosis factor therapy. Optionally, the subject had a DAS28 score of >5.1 when originally assessed for anti-tumour necrosis factor therapy of rheumatoid arthritis. Optionally, the subject had a DAS28 score of greater than 5.1 when originally assessed for anti-tumour necrosis factor therapy of rheumatoid arthritis.

Optionally, the subject has a DAS28 score of >3.2. Optionally, the subject has a DAS28 score of greater than 3.2. Optionally, the subject had a DAS28 score of >3.2 when originally assessed for anti-tumour necrosis factor therapy. Optionally, the subject had a DAS28 score of greater than 3.2 when originally assessed for anti-tumour necrosis factor therapy. Optionally, the subject had a DAS28 score of >3.2 when originally assessed for anti-tumour necrosis factor therapy of rheumatoid arthritis. Optionally, the subject had a DAS28 score of greater than 3.2 when originally assessed for anti-tumour necrosis factor therapy of rheumatoid arthritis.

Optionally, the subject fulfils the American College of Rheumatology 1987 revised criteria for rheumatoid arthritis diagnosis. Optionally, the subject is assigned to anti-tumour necrosis factor therapy as part of routine clinical practice. Optionally, the subject fulfils the British Society for Rheumatology criteria for anti-tumour necrosis factor therapy. Optionally, the subject has failed at least one disease-modifying anti-rheumatic drug (DMARD). Optionally, the subject has failed at least two disease-modifying anti-rheumatic drugs (DMARDs).

Optionally, the method comprises a polymerase chain reaction method.

Optionally, the method comprises a polymerase chain reaction-sequence-specific oligonucleotide probe method.

Optionally, the biological sample substantially comprises a blood sample.

Optionally, the biological sample substantially comprises a tissue sample.

Optionally, there is provided a method for predicting responsiveness to anti- tumour necrosis factor therapy of rheumatoid arthritis in a subject, the method comprising the steps of:

a) Providing a biological sample;

b) detecting the presence, absence, or quantitative level of a first marker or an expression product thereof, wherein the first marker is at the HLA-DRB1 gene;

c) detecting the presence, absence, or quantitative level of a second marker, or an expression product thereof, wherein the second marker is at the CD226 gene;

d) correlating the presence, absence, or quantitative level of the first marker and the presence, absence, or quantitative level of the second marker; to the predicted responsiveness to anti-tumour necrosis factor therapy of rheumatoid arthritis in the subject.

Optionally, the first marker comprises at least part of a nucleic acid sequence selected from the group comprising: the nucleic acid sequence of the HLA-DRB1*0404 allele, and the nucleic acid sequence of SEQ ID NO: 1.

Optionally, the first marker comprises a nucleic acid sequence selected from the group comprising: the nucleic acid sequence of the HLA-DRB1*0404 allele, and the nucleic acid sequence of SEQ ID NO: 1.

Optionally, the HLA-DRB1 gene is the National Center for Biotechnology Information (NCBI) Gene ID: 3123. Optionally the HLA-DRB1*0404 allele is the NCBI GenBank accession number AF352292, version number AF352292.1.

Optionally, the second marker comprises a nucleic acid sequence selected from the group comprising: the nucleic acid sequence of the rs763361 single nucleotide polymorphism, and the nucleic acid sequence of SEQ ID NO: 2.

Optionally, the CD226 gene is the National Center for Biotechnology Information (NCBI) Gene ID: 10666.

Optionally, the rs763361 single nucleotide polymorphism is a CD226 gene single nucleotide polymorphism represented by the National Center for Biotechnology Information (NCBI) Reference SNP number rs763361. Optionally, the rs763361 single nucleotide polymorphism is a thymine (T) to cytosine (C) single nucleotide polymorphism (SNP) in the CD226 gene. Optionally, the rs763361 single nucleotide polymorphism is a thymine (T) to cytosine (C) single nucleotide polymorphism (SNP) in the CD226 gene at the NCBI Reference SNP anchor position chr18:69864406 (GRCh38.p12).

Optionally, absence of the second marker is homozygous absence of the second marker.

Optionally, the presence of the first marker indicates that the subject is predicted to be responsive to anti-tumour necrosis factor therapy of rheumatoid arthritis.

Optionally, the absence of the second marker indicates that the subject is predicted to be responsive to anti-tumour necrosis factor therapy of rheumatoid arthritis.

Optionally, the method further comprises the step of evaluating the DAS28 score of the subject.

Optionally, the method comprises a polymerase chain reaction method. Optionally, the method comprises a polymerase chain reaction-sequence-specific oligonucleotide probe.

Optionally, the method comprises a biochip array method.

Optionally, the anti- tumour necrosis factor therapy of rheumatoid arthritis comprises a drug selected from the group comprising: adalimumab, etanercept, infliximab, certolizumab, certolizumab pegol, golimumab, and combinations thereof.

Optionally, the anti-tumour necrosis factor therapy comprises a drug selected from the group comprising: adalimumab, etanercept, infliximab, certolizumab, certolizumab pegol, golimumab, and combinations thereof.

Optionally, detecting step (b) comprises: contacting the sample with at least one primer having a nucleic acid sequence defined by at least one of: SEQ ID NO: 3, 4, and the reverse complement each thereof; under conditions suitable for a polymerase chain reaction.

Optionally, detecting step (c) comprises: contacting the sample with at least one primer having a nucleic acid sequence defined by at least one of: SEQ ID NO: 5, 6, and the reverse complement each thereof; under conditions suitable for a polymerase chain reaction.

Optionally, the biological sample substantially comprises a blood sample.

Optionally, there is provided an in vitro method for predicting responsiveness to anti-tumour necrosis factor therapy of rheumatoid arthritis in a subject comprising any of the above-described methods, wherein the presence of the first marker and the absence of the second marker indicates that the subject is predicted to be responsive to anti-tumour necrosis factor therapy of rheumatoid arthritis.

According to a second aspect of the present invention, there is provided an anti-tumour necrosis factor rheumatoid arthritis therapy response prediction kit, comprising at least one primer or probe having a nucleic acid sequence defined by any of SEQ ID NO: 3, 4, 5, and 6.

Optionally, the kit further comprises instructions for use.

Optionally, the kit further comprises a solid support.

Optionally, there is provided an anti- tumour necrosis factor rheumatoid arthritis therapy response prediction kit, comprising at least one primer or probe having a nucleic acid sequence defined by any of SEQ ID NO: 3, 4, 5, and 6.

Optionally, the kit further comprises a solid support, optionally further comprising instructions for use.

### BRIEF DESCRIPTION OF THE DRAWINGS

FIG. 1

Charts showing significant factors associated with baseline DAS28 score. A Box plot of baseline DAS28 scores for RA patients with the HLA-DRB1 0404 allele absent or present. Central bar represents mean, outer box standard error and error bars standard deviation of grouping. B Dot plot correlation of baseline DAS28 scores for RA patients versus baseline health assessment questionnaire score.

FIG. 2

Charts showing association between genetic and clinical factors and change in DAS28 score after 6 months treatment. A Association between baseline DAS28 score and delta DAS28. B Association between baseline DMARD use and delta DAS28. 0, none used; 1, DMARD used. C Association between baseline HAQ score and delta DAS28. D Association between gender and delta DAS28. E Association between HLA-DRB1 0404 genotypes and delta DAS28. 0, absent; 1, present. F Association between binary CD226 and delta DAS28. wt, wildtype TT; combined, CT and CC carriers.

FIG. 3

Charts showing the model of combined effects of HLA-DRB1*0404 allele and CD226 SNP rs763361 on change in DAS28 score after 6 months treatment. A Non-interaction plot SNP genotypes: 11, wildtype; 12, heterozygous carrier; 22 homozygous carrier of CD226 SNP. B Effect of CD226 rs763361SNP presence upon change in DAS28 ESR score after 6 months of anti-TNF use; 11 is homozygous wildtype TT, 12 is CT and 22 is CC genotype. C Combined HLA-DRB1*0404 and binary CD226 effect plot. For HLA-DRB1*0404 allele, 0 is absent, 1 is present. For CD226 rs763361 SNP, 11 is homozygous wildtype TT, 12 is CT and 22 is CC genotype.

FIG. 4

Charts showing that there are significant differences among the 6 genotypes of CD226-HLA0404 combinations. Homozygous CD226 and presence of HLA0404 allele represents the most responsive genotype, significantly better than most of the other 5 combinations. A recDAST0 effect plot. B HLA040*CD226 effect plot.

FIG. 5

Charts showing that the homozyous genotype 11 of CD226 conforms to the best response. Patients with Genotypes CD226:12 or CD226:22 have significantly worse response than genotype CD226:11. A recDAST0 effect plot. B CD226 effect plot.

FIG. 6

Charts showing that in patients who received etanercept treatment, Genotype 12 of CHUK conforms to the best response, which is significantly better than the homozygous genotype CHUK:11. A recDAST0 effect plot. B CHUK effect plot.

FIG. 7

Charts showing influence of various factors on absolute changes in disease activity score over the biologic treatment period A recDAST0 effect plot. B HAQT0 effect plot. C DMARDT0*Gender effect plot. D DMARDT0*HLA4040 effect plot. E DMARDT0*CD226 binary effect plot. F Gender*HLA4040 effect plot. G Gender*CD226 binary effect plot. H HLA4040*CD226 binary effect plot.

### EXAMPLES

The inventors conducted a study to investigate whether specific killer-cell immunoglobulin-like receptor (KIR) and human leukocyte antigen (HLA) gene haplotypes and several promising genetic variants could predict response to anti-TNF treatment in a cohort of biologic naive RA patients in the UK. The purpose of this study was to determine if baseline genetic variants of PTPRC, AFF3, myD228, CHUK, MTHFR1, MTHFR2, CD226 and a number of KIR and HLA alleles could predict response to anti-tumour necrosis factor (TNF) treatment in rheumatoid arthritis (RA) patients.

The PTPRC gene encodes the protein tyrosine phosphatase, receptor type C protein. The AFF3 gene encodes the AF4/FMR2 family member 3 protein. The myD228 gene encodes the myeloid differentiation primary response gene 88 protein. The CHUKgene encodes the conserved helix-loop-helix ubiquitous kinase protein. The MTHFR1 and MTHFR2 genes encode the methylenetetrahydrofolate reductase 1 and methylenetetrahydrofolate reductase 2 proteins, respectively. The CD226 gene encodes the Cluster of Differentiation 226 protein.

### Methods

### Patient Selection

The following criteria were used for the selection of patients for the current study: (1) fulfilled the American College of Rheumatology 1987 revised criteria for RA diagnosis, (2) assigned to anti-TNF-α treatment as part of routine clinical practice, (3) fulfilled the British Society for Rheumatology (BSR) criteria for anti-TNF-α therapy and had failed at least two disease-modifying anti-rheumatic drugs (DMARDs), (4) had a 28 joint disease activity score (DAS28) score of >5.1 when originally assessed for treatment, (5) reached 6 months of follow-up. Patients who stopped anti-TNF-α temporarily during first six months and patients who discontinued therapy prior to the 6 month follow up for reasons other than inefficacy were excluded. Two hundred and thirty eight eligible RA patients treated with anti-TNF drugs were recruited from rheumatology biologic clinics at Altnagelvin Hospital, Londonderry and Musgrave Park Hospital, Belfast, both in Northern Ireland.

### Patient Recruitment, Sample Collection, Clinical Information

Eligible patients from each hospital were invited to take part in the study by mailing patient information sheets, explaining the study and patient involvement, a minimum of 48 hours before a routine care appointment. Additional blood samples were obtained from consenting patients who were either about to commence, or had been on an anti-TNF-α treatment in the past. Blood samples were processed by a silica based extraction kit to isolate genomic DNA (such as a DNeasy® Blood & Tissue Kit, QIAGEN® Inc.). DNA samples were aliquoted and stored at −80° C. until analysis. Clinical and demographic information was extracted from medical records and clinic databases after consent. Disease activity was compiled for baseline and 6 months of treatment with anti-TNF-α, using the 28-joint disease activity score-erythrocyte sedimentation rate (DAS28-ESR). Following 6 months of treatment, the patients were assigned a moderate responder, good responder or non-responder status, according to the EULAR criteria. The main demographic and clinical features of the patients are shown in Table 1. Office for Research Ethics Committees Northern Ireland (ORECNI) approval was obtained for the study.

TABLE 1
Patient characteristics by EULAR response class
after six months of anti-TNF treatment

| | Non- | Moderate | Good | Com- | |
|---|---|---|---|---|---|
| Cohort | responders | responders | responders | bined | |
| characteristics | (n = 60) | (n = 87) | (n = 93) | (n = 240) | P-value |
| Gender, | 44 | 70 | 66 | 180 | 0.320 |
| female, n (%) | (73.3) | (80.5) | (71.0) | (75.0) | |
| Age at | 52.8 | 54.0 | 54.3 | 53.8 | 0.740 |
| baseline, | (13.1) | (11.9) | (12.0) | (12.2) | |
| mean (s.d.), | | | | | |
| years | | | | | |
| Concurrent | 51 | 73 | 82 | 206 | 0.700 |
| DMARDs, | (85.0) | (83.9) | (88.2) | (85.8) | |
| n (%) | | | | | |
| DAS28 score | 4.9 | 5.9 | 5.3 | 5.4 | 3.60E−05 |
| at baseline, | (1.3) | (1.1) | (1.1) | (1.2) | |
| mean (s.d.) | | | | | |
| DAS28 score | 5.3 | 4.1 | 2.1 | 3.8 | 1.60E−40 |
| at outcome, | (1.2) | (0.8) | (0.8) | (1.6) | |
| mean (s.d.) | | | | | |
| Change in | 0.4 | −1.8 | −3.1 | −1.6 | 4.10E−45 |
| DAS28 | (1.0) | (0.8) | (1.0) | (1.6) | |
| score, | | | | | |
| mean (s.d.) | | | | | |
| Baseline TJC, | 9.2 | 12.3 | 11.6 | 11.2 | 0.027 |
| mean (s.d.) | (6.4) | (7.4) | (6.7) | (7.0) | |
| Baseline SJC, | 5.7 | 8.9 | 8.9 | 8.1 | 8.50E−04 |
| mean (s.d.) | (5.0) | (5.7) | (5.7) | (5.7) | |
| Baseline | 15.6 | 25.3 | 16.1 | 19.2 | 0.063 |
| CRP, | (23.1) | (35.6) | (21.7) | (28.0) | |
| mean (s.d.) | | | | | |
| Baseline | 1.5 (0.9), | 1.5 (0.9), | 1.6 (0.8), | 1.6 (0.8), | 0.850 |
| HAQ, | 45 | 55 | 57 | 157 | |
| mean (s.d.), n | | | | | |
| Outcome | 8.8 | 5.4 | 1.4 | 4.8 | 1.7E−16 |
| TJC, | (6.5) | (4.8) | (2.6) | (5.5) | |
| mean (s.d.) | | | | | |
| Outcome | 5.7 | 3.1 | 0.9 | 3.0 | 2.03E−14 |
| SJC, | (4.8) | (3.4) | (1.3) | (3.8) | |
| mean (s.d.) | | | | | |
| Outcome | 16.7 | 8.2 | 4.6 | 9.1 | 6.55E−06 |
| CRP, | (22.9) | (10.5) | (6.2) | (14.6) | |
| mean (s.d.) | | | | | |
| Outcome | 1.3 (0.9), | 1.5 (0.9), | 1.0 (0.8), | 1.3 (0.9), | 0.0415 |
| HAQ, | 34 | 39 | 31 | 104 | |
| mean (s.d.), n | | | | | |

All values are mean with standard deviation (s.d.), or percentage where indicated (%).
DAS28 = 28 joint disease activity score;
DMARD = disease modifying anti-rheumatic drug;
HAQ = health assessment questionnaire;
CRP = C-reactive protein;
SJC = swollen joint count;
TJC = tender joint count.

### Genotyping

All genotyping was performed by biochip array technology (such as custom Rheumastrat™ biochip array technology (Evidence Analyser™, Randox Laboratories Ltd.). Genotyping was confirmed by the polymerase chain reaction-sequence-specific oligonucleotide probe (PCR-SSOP) method described by McGeough C M et al. (2012), and Middleton D et al. (2005). Positive controls of known KIR, HLA or single nucleotide polymorphism (SNP) genotype, were included in the typing procedure. DNA was typed for the presence or absence of previous response associated framework KIR genes: KIR2DS2 (activator) and KIR2DL2 (inhibitor). HLA-DRB1 typing was performed on the following shared epitope alleles: *03, *0101, *1001, *0401, *0104 and *0404. A modified version of the HLA-C typing method was used to define the HLA-C1 and C2 groups using probe C293 and C291, respectively.

Single nucleotide polymorphisms previously published as associated with therapeutic response and disease severity were typed for the following gene loci (in brackets): HLA-DR/BTNL2 (rs1980493), protein tyrosine phosphatase, receptor type C (PTPRC) (rs10919563), AFF3 (rs10865035), CD226 (rs763361), myD88 (rs7744), CHUK (rs11591741), methylenetetrahydrofolate reductase 1 (MTHFR1) (rs1801133), methylenetetrahydrofolate reductase 2 (MTHFR2) (rs1801131).

### Statistical Methods and Analysis

The significance of the differences in proportions of responders and non-responders exhibiting a specific genotype was assessed using Fisher's exact test. For the numeric measures including DAS28 scores at baseline, DAS28 score changes at month 6 (ΔDAS28), Health Assessment

Questionnaire scores at baseline, t-based statistics were used to assess the difference between two means, one-way ANOVA for multiple means, and Pearson correlation for assessing correlations between numerical variables. A series of systematic linear regression analyses were used to construct a most appropriate models consisting of significant predictors (detailed in the Results section). All tests were two-sided unless otherwise stated. Where applicable, adjustments for multiple testing were made using Holm's method.

### Results

### Patient Demographics

There was no significant difference among the three response groups (ie, good, moderate, and non-responder) of patients with respect to the distribution of age (p=0.74, one-way ANOVA was used for age and other numeric variables listed in Table 1), gender (p=0.32, Chi-squared test was for gender and other categorical variables in Table 1) or DMARD use (p=0.70) (Table 1 study cohort characteristics). Baseline tender and swollen joint counts were significantly lower in non-responders, whereas baseline C-reactive protein (CRP) was significantly elevated in moderate responders; both differences were statistically significant (p=0.027, p=8.5E-04, p=0.063 respectively). The mean change in DAS28 differed significantly between each EULAR response group, with non-responders at 0.4±1.0, moderate responders −1.8±0.8 and good responders −3.1±1.0 (p=4.10E-45). For patients who had laboratory data available, 149 or 73.8% of those tested were rheumatoid factor positive at the start of the study and 94 or 72.9% of those tested were anti-cyclic citrullinated peptide (anti-CCP) antibody positive.

236 patients received a combination of conventional DMARD and anti-TNF-α drugs. Of the five anti-TNF drugs prescribed in the study population, adalimumab was prescribed for 119 (50.4%), etanercept for 70 (29.7%), infliximab for 35 (14.8%), certoluzimab for 8 (3.4%) and golimumab for 4 (1.69%) (Table 2 of anti-TNF drugs prescribed across study cohort). There was no significant difference among the five anti-TNF-α drugs in terms of the treatment outcome in those patients who received conventional DMARD (ANOVA, p=0.093).

TABLE 2
Anti-TNF treatment profile of study cohort

| | | | | HLA- | | | |
|---|---|---|---|---|---|---|---|
| | | RF | Anti-CCP | DRB1 | CD226 | CD226 | CD226 |
| | | positive | positive | 0404 | (TC) | (CC) | total |
| Adalimumab | n positive | 77 | 50 | 25 | 62 | 36 | 98 |
| | n available | 100 | 67 | 119 | 119 | 119 | 119 |
| | % | 77.0% | 74.6% | 21.0% | 52.1% | 30.3% | 82.4% |
| Etanercept | n positive | 47 | 34 | 9 | 32 | 25 | 57 |
| | n available | 64 | 44 | 70 | 70 | 70 | 70 |
| | % | 73.4% | 77.3% | 12.9% | 45.7% | 35.7% | 81.4% |
| Infliximab | n positive | 19 | 7 | 12 | 21 | 7 | 28 |
| | n available | 28 | 12 | 35 | 35 | 35 | 35 |
| | % | 67.9% | 58.3% | 34.3% | 60.0% | 20.0% | 80.0% |
| Certoluzimab | n positive | 5 | 3 | 3 | 6 | 2 | 8 |
| | n available | 8 | 5 | 8 | 8 | 8 | 8 |
| | % | 62.5% | 60.0% | 37.5% | 75.0% | 25.0% | 100.0% |
| Golimumab | n positive | 1 | 0 | 1 | 4 | 0 | 4 |
| | n available | 2 | 1 | 4 | 4 | 4 | 4 |
| | % | 50.0% | 0.0% | 25.0% | 100.0% | 0.0% | 100.0% |
| Combined | n positive | 149 | 94 | 50 | 125 | 70 | 195 |
| | n available | 202 | 129 | 236 | 236 | 236 | 236 |
| | % | 73.8% | 72.9% | 21.2% | 53.0% | 29.7% | 82.6% |

RF, rheumatoid factor;
anti-CCP, anti-cyclic citrullinated peptide;
hetero, heterozygous carrier;
homo, homozygous carrier

### Baseline Characteristics (Gender, HAQ and Genotype vs Baseline DAS28)

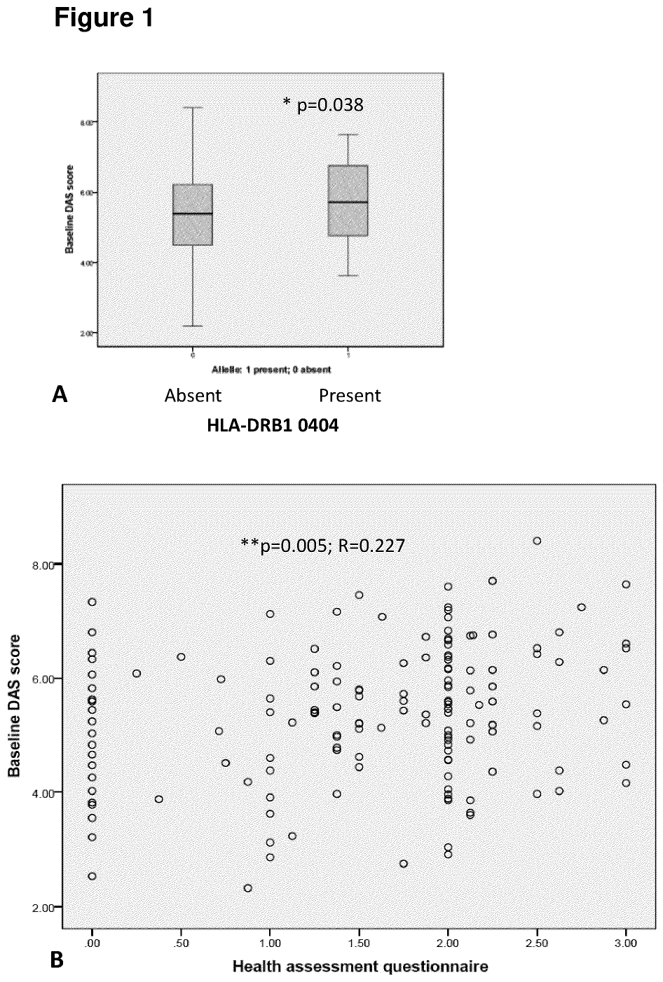

Baseline DAS28 was found to be significantly increased in patients with HLA-DRB1*0404 haplotype (p=0.038; absent mean DAS28 5.30±1.235; present mean DAS28 5.75±1.151; FIG. 1A, Table 4). Gender and baseline DMARD both exerted an effect on change in DAS28 over 6 months of treatment (ΔDAS28), but did not have any significant association in the study RA population (Table 3). Since strength of association of a particular genotype with ΔDAS28 could be influenced by gender, baseline DAS28 and concurrent DMARD use, adjustments considering these factors were necessary in later predictive model tests. As has been observed in previous studies, baseline DAS28 is significantly correlated with baseline health assessment questionnaire (HAQ) (p=0.005; R=0.227; n=154; FIG. 1B).

TABLE 3
Association of baseline clinical factors with change in
DAS28 score over 6 months treatment.

| | | Group | Group | Group Mean | p |
|---|---|---|---|---|---|
| Factor | Group | No.s | % | delta DAS28 | value |
| Gender | Male | 59 | 24.8% | −1.70 | 0.127 |
| | Female | 179 | 75.2% | −1.81 | |
| Baseline | No | 33 | 13.9% | −1.71 | 0.694 |
| DMARD | Yes | 205 | 86.1% | −1.80 | |

TABLE 4
Association of factors with baseline DAS28 score

| | | Group | Group | Group Mean | p |
|---|---|---|---|---|---|
| Factor | Group | No.s | % | Baseline DAS28 | value |
| Gender | Male | 47 | 23.9% | 5.16 | 0.127 |
| | Female | 150 | 76.1% | 5.47 | |
| Baseline | No | 21 | 10.7% | 5.50 | 0.694 |
| DMARD | Yes | 176 | 89.3% | 5.39 | |
| KIR2DL2 | 0 | 80 | 40.6% | 5.53 | 0.203 |
| | 1 | 117 | 59.4% | 5.31 | |
| KIR2DS2 | 0 | 76 | 38.6% | 5.53 | 0.233 |
| | 1 | 121 | 61.4% | 5.31 | |
| HLAC1 | 0 | 25 | 12.7% | 5.19 | 0.362 |
| | 1 | 172 | 87.3% | 5.43 | |
| HLAC2 | 0 | 86 | 43.7% | 5.58 | 0.070 |
| | 1 | 111 | 56.3% | 5.26 | |
| HLA03 | 0 | 155 | 78.7% | 5.42 | 0.658 |
| | 1 | 42 | 21.3% | 5.32 | |
| HLA0101 | 0 | 138 | 70.1% | 5.38 | 0.753 |
| | 1 | 59 | 29.9% | 5.44 | |
| HLA0401 | 0 | 99 | 50.3% | 5.41 | 0.907 |
| | 1 | 98 | 49.7% | 5.39 | |
| HLA0404 | 0 | 155 | 78.7% | 5.30 | 0.038 |
| | 1 | 42 | 21.3% | 5.75 | |
| HLA1001 | 0 | 188 | 95.4% | 5.37 | 0.196 |
| | 1 | 9 | 4.6% | 5.92 | |
| HLADRBTNL | 11 | 139 | 70.6% | 5.39 | 0.932 |
| | 12 | 52 | 26.4% | 5.41 | |
| | 22 | 6 | 3.0% | 5.58 | |
| PTPRC | 11 | 155 | 78.7% | 5.41 | 0.353 |
| | 12 | 41 | 20.8% | 5.38 | |
| | 22 | 1 | 0.5% | 3.64 | |
| AFF | 11 | 50 | 25.4% | 5.42 | 0.981 |
| | 12 | 108 | 54.8% | 5.38 | |
| | 22 | 39 | 19.8% | 5.40 | |
| CD226 | 11 | 30 | 15.2% | 5.42 | 0.921 |
| | 12 | 108 | 54.8% | 5.37 | |
| | 22 | 59 | 29.9% | 5.44 | |
| myD | 11 | 134 | 68.0% | 5.48 | 0.307 |
| | 12 | 58 | 29.4% | 5.25 | |
| | 22 | 5 | 2.5% | 4.85 | |
| CHUK | 11 | 61 | 31.0% | 5.22 | 0.403 |
| | 12 | 89 | 45.2% | 5.48 | |
| | 22 | 47 | 23.9% | 5.46 | |
| MTHFR1 | 11 | 88 | 44.7% | 5.43 | 0.928 |
| | 12 | 82 | 41.6% | 5.38 | |
| | 22 | 27 | 13.7% | 5.34 | |
| MTHFR2 | 11 | 93 | 47.2% | 5.30 | 0.567 |
| | 12 | 85 | 43.1% | 5.50 | |
| | 22 | 19 | 9.6% | 5.44 | |

Allele genotypes: 1 = present; 0 = absent.
SNP genotypes: 11 = wildtype; 12 = heterozygous carrier; 22 = homozygous carrier.

### Individual Factor Association with Response to Anti-TNF Treatment

Using the EULAR classification system none of the shared epitope alleles were significantly associated with response to anti TNF though HLA-DRB1*0404 came close at p=0.059 (Table 5). The MTHFR1 SNP was significantly associated with the EULAR response, p=0.044, though not significant for CD226, p=0.202.

TABLE 5
Association of genetic factors with EULAR response

| | | EULAR RESPONSE, | | | | | | |
|---|---|---|---|---|---|---|---|---|
| | | genotype n (%) | | | | | | |
| | Genotype | NON | | MODERATE | | GOOD | | |
| Factor | Group | n | % | n | % | n | % | p value |
| KIR2DL2 | 0 | 23 | 22.3% | 42 | 40.8% | 38 | 36.9% | 0.601 |
| | 1 | 37 | 27.4% | 48 | 35.6% | 50 | 37.0% | |
| KIR2DS2 | 0 | 22 | 22.2% | 40 | 40.4% | 37 | 37.4% | 0.635 |
| | 1 | 38 | 27.3% | 50 | 36.0% | 51 | 36.7% | |
| HLAC1 | 0 | 8 | 27.6% | 8 | 27.6% | 13 | 44.8% | 0.463 |
| | 1 | 52 | 24.9% | 82 | 39.2% | 75 | 35.9% | |
| HLAC2 | 0 | 25 | 23.4% | 44 | 41.1% | 38 | 35.5% | 0.626 |
| | 1 | 35 | 26.7% | 46 | 35.1% | 50 | 38.2% | |
| HLA03 | 0 | 45 | 24.2% | 68 | 36.6% | 73 | 39.2% | 0.388 |
| | 1 | 15 | 28.8% | 22 | 42.3% | 15 | 28.8% | |
| HLA0101 | 0 | 41 | 24.4% | 65 | 38.7% | 62 | 36.9% | 0.877 |
| | 1 | 19 | 27.1% | 25 | 35.7% | 26 | 37.1% | |
| H LA0401 | 0 | 33 | 26.6% | 44 | 35.5% | 47 | 37.9% | 0.728 |
| | 1 | 27 | 23.7% | 46 | 40.4% | 41 | 36.0% | |
| H LA0404 | 0 | 51 | 27.3% | 74 | 39.6% | 62 | 33.2% | 0.060 |
| | 1 | 9 | 17.6% | 16 | 31.4% | 26 | 51.0% | |
| HLA1001 | 0 | 57 | 24.9% | 87 | 38.0% | 85 | 37.1% | 0.849 |
| | 1 | 3 | 33.3% | 3 | 33.3% | 3 | 33.3% | |
| HLADRBTNL | 11 | 40 | 23.5% | 62 | 36.5% | 68 | 40.0% | 0.488 |
| | 12 | 18 | 29.5% | 24 | 39.3% | 19 | 31.1% | |
| | 22 | 2 | 28.6% | 4 | 57.1% | 1 | 14.3% | |
| PTPRC | 11 | 41 | 22.2% | 75 | 40.5% | 69 | 37.3% | 0.188 |
| | 12 | 19 | 37.3% | 14 | 27.5% | 18 | 35.3% | |
| | 22 | 0 | 0.0% | 1 | 50.0% | 1 | 50.0% | |
| AFF | 11 | 41 | 22.2% | 75 | 40.5% | 69 | 37.3% | 0.886 |
| | 12 | 19 | 37.3% | 14 | 27.5% | 18 | 35.3% | |
| | 22 | 0 | 0.0% | 1 | 50.0% | 1 | 50.0% | |
| CD226 | 11 | 7 | 17.1% | 13 | 31.7% | 21 | 51.2% | 0.202 |
| | 12 | 36 | 28.6% | 51 | 40.5% | 39 | 31.0% | |
| | 22 | 17 | 23.9% | 26 | 36.6% | 28 | 39.4% | |
| myD | 11 | 44 | 27.3% | 64 | 39.8% | 53 | 32.9% | 0.220 |
| | 12 | 13 | 18.6% | 24 | 34.3% | 33 | 47.1% | |
| | 22 | 3 | 42.9% | 2 | 28.6% | 2 | 28.6% | |
| CHUK | 11 | 22 | 27.8% | 31 | 39.2% | 26 | 32.9% | 0.834 |
| | 12 | 23 | 22.1% | 40 | 38.5% | 41 | 39.4% | |
| | 22 | 15 | 27.3% | 19 | 34.5% | 21 | 38.2% | |
| MTHFR1 | 11 | 27 | 25.0% | 47 | 43.5% | 34 | 31.5% | 0.045 |
| | 12 | 22 | 22.2% | 30 | 30.3% | 47 | 47.5% | |
| | 22 | 11 | 35.5% | 13 | 41.9% | 7 | 22.6% | |
| MTHFR2 | 11 | 32 | 27.4% | 44 | 37.6% | 41 | 35.0% | 0.745 |
| | 12 | 21 | 21.6% | 36 | 37.1% | 40 | 41.2% | |
| | 22 | 7 | 29.2% | 10 | 41.7% | 7 | 29.2% | |

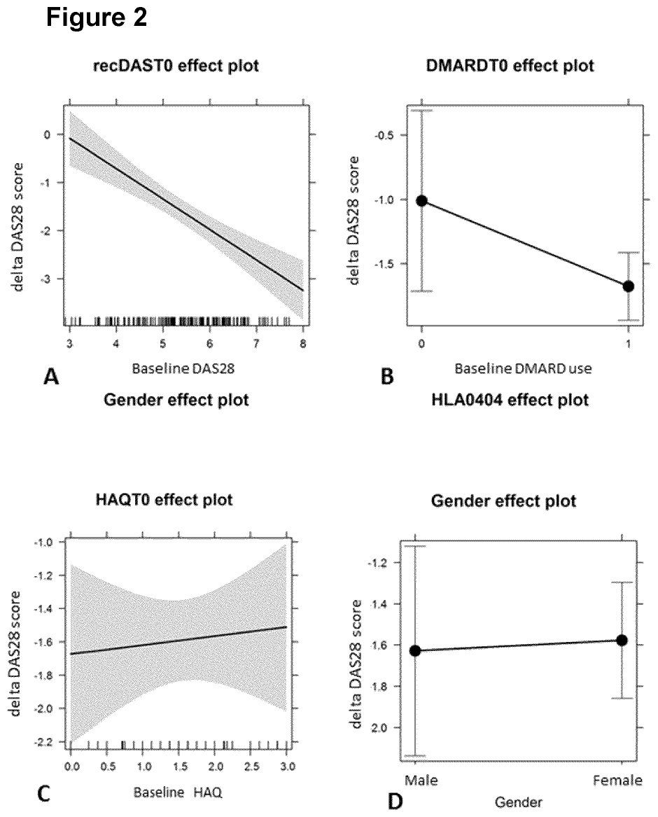

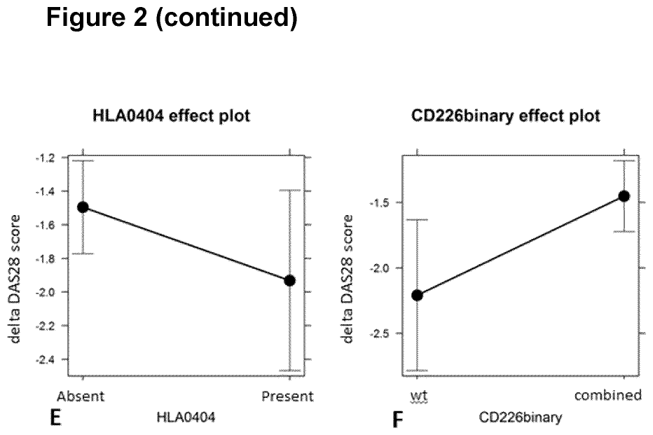

The associations between presence or absence of individual alleles on 4DAS28 were investigated (Table 6; FIG. 2). Notably, presence of the HLA-DRB1*0404 allele was significantly associated with a larger drop in mean ΔDAS28 of −2.22 , compared to absence mean ΔDAS28 of -1.67 (p=0.0329). CD226 SNP was the only gene variant significantly associated with ΔDAS28 (p=0.0287) (Table 6). Relative to the CD226 homozygous wild type (TT) mean ΔDAS28 of −2.35 after anti-TNF treatment, carriers of the CD226 polymorphism had reduced responses, with the heterozygous carrier genotype (TC), mean ΔDAS28 of −1.57, and homozygous genotype (CC), mean ΔDAS28 of −1.85.

TABLE 6
Association of genetic factors with change in DAS28
score over 6 months treatment

| | | Group | Group | Group Mean | Std. | p |
|---|---|---|---|---|---|---|
| Factor | Group | No.s | % | delta DAS28 | Dev. | value |
| KIR2DL2 | 0 | 103 | 43.3% | −1.89 | | 0.398 |
| | 1 | 135 | 56.7% | −1.71 | | |
| KIR2DS2 | 0 | 99 | 41.6% | −1.91 | | 0.320 |
| | 1 | 139 | 58.4% | −1.70 | | |
| HLAC1 | 0 | 29 | 12.2% | −1.76 | | 0.929 |
| | 1 | 209 | 87.8% | −1.79 | | |
| HLAC2 | 0 | 107 | 45.0% | −1.84 | | 0.684 |
| | 1 | 131 | 55.0% | −1.75 | | |
| HLA03 | 0 | 186 | 78.2% | −1.86 | | 0.178 |
| | 1 | 52 | 21.8% | −1.51 | | |
| HLA0101 | 0 | 168 | 70.6% | −1.76 | | 0.708 |
| | 1 | 70 | 29.4% | −1.85 | | |
| HLA0401 | 0 | 124 | 52.1% | −1.83 | | 0.653 |
| | 1 | 114 | 47.9% | −1.74 | | |
| HLA0404 | 0 | 187 | 78.6% | −1.67 | | 0.033 |
| | 1 | 51 | 21.4% | −2.22 | | |
| HLA1001 | 0 | 229 | 96.2% | −1.80 | | 0.489 |
| | 1 | 9 | 3.8% | −1.41 | | |
| HLADRBTNL | 11 | 170 | 86.3% | −1.89 | | 0.329 |
| | 12 | 61 | 31.0% | −1.55 | | |
| | 22 | 7 | 3.6% | −1.42 | | |
| PTPRC | 11 | 185 | 93.9% | −1.85 | | 0.281 |
| | 12 | 51 | 25.9% | −1.53 | | |
| | 22 | 2 | 1.0% | −3.00 | | |
| AFF | 11 | 59 | 29.9% | −1.61 | | 0.588 |
| | 12 | 126 | 64.0% | −1.81 | | |
| | 22 | 53 | 26.9% | −1.93 | | |
| CD226 | 11 | 41 | 20.8% | −2.35 | | 0.029 |
| | 12 | 126 | 64.0% | −1.57 | | |
| | 22 | 71 | 36.0% | −1.85 | | |
| myD | 11 | 161 | 81.7% | −1.77 | | 0.916 |
| | 12 | 70 | 35.5% | −1.84 | | |
| | 22 | 7 | 3.6% | −1.61 | | |
| CHUK | 11 | 79 | 40.1% | −1.65 | | 0.479 |
| | 12 | 104 | 52.8% | −1.93 | | |
| | 22 | 55 | 27.9% | −1.71 | | |
| MTHFR1 | 11 | 108 | 54.8% | −1.71 | | 0.116 |
| | 12 | 99 | 50.3% | −2.01 | | |
| | 22 | 31 | 15.7% | −1.34 | | |
| MTHFR2 | 11 | 117 | 59.4% | −1.67 | | 0.341 |
| | 12 | 97 | 49.2% | −1.97 | | |
| | 22 | 24 | 12.2% | −1.58 | | |

### Multi-Factor Regression Models

There were previous reports that ΔDAS28 could be influenced by gender, baseline DAS28 and concurrent DMARD use, and even HAQ, we thus investigated whether these four factors are significantly associated with ΔDAS28 in our cohort of patients. We fit a linear regression model using ΔDAS28 as dependent variable, and the four factors mentioned here as independent variables, at first considering the interaction between the two categorical variables gender and concurrent DMARD use. However the gender-DMARD interaction term was found to be non-significant (p-value=0.26), subsequently a simplified regression model without interaction terms was fitted. The results of this regression analysis are shown in Table 7. As can be seen from this table, gender (p=0.99) and HAQ (p=0.52) were not significantly associated with ΔDAS28. So these two terms were also excluded from all subsequent analysis.

TABLE 7
The linear model with four potential predictors

| Variable | Estimate | Std. Error | t value | Pr(>\|t\|) |
|---|---|---|---|---|
| (Intercept) | 2.48595 | 0.680512 | 3.653056 | 0.000358 |
| Baseline | −0.66396 | 0.110541 | −6.0065 | 1.39E−08 |
| DAS28 | | | | |
| Concomittant | −0.76153 | 0.386531 | −1.97016 | 0.050674 |
| DMARD | | | | |
| Baseline | 0.10124 | 0.157878 | 0.641258 | 0.52234 |
| HAQ | | | | |
| Gender | 0.004174 | 0.301256 | 0.013856 | 0.988964 |

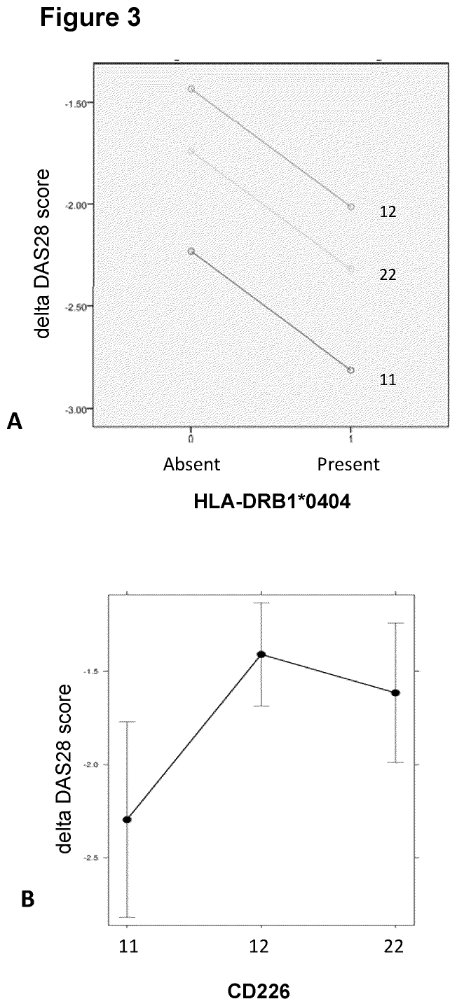

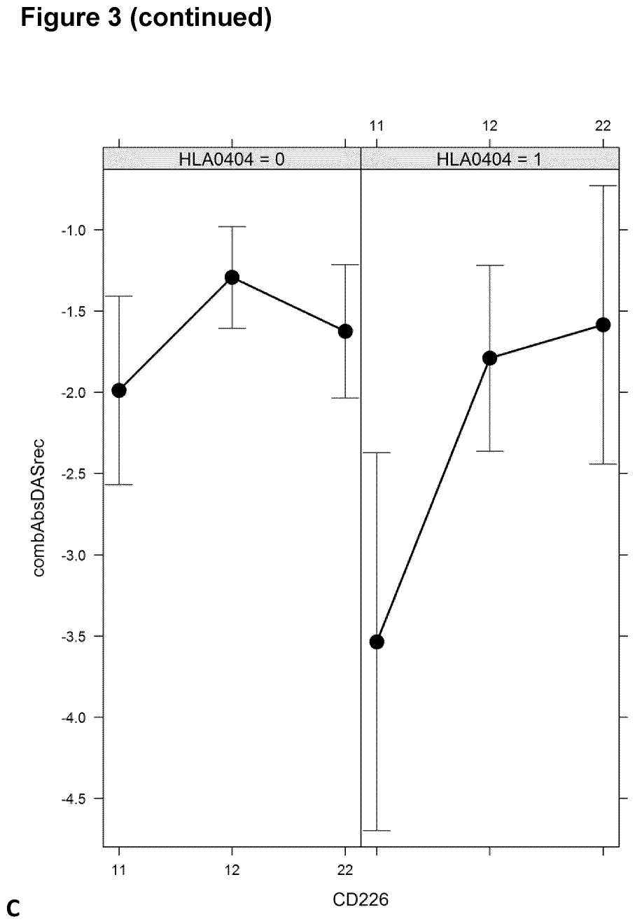

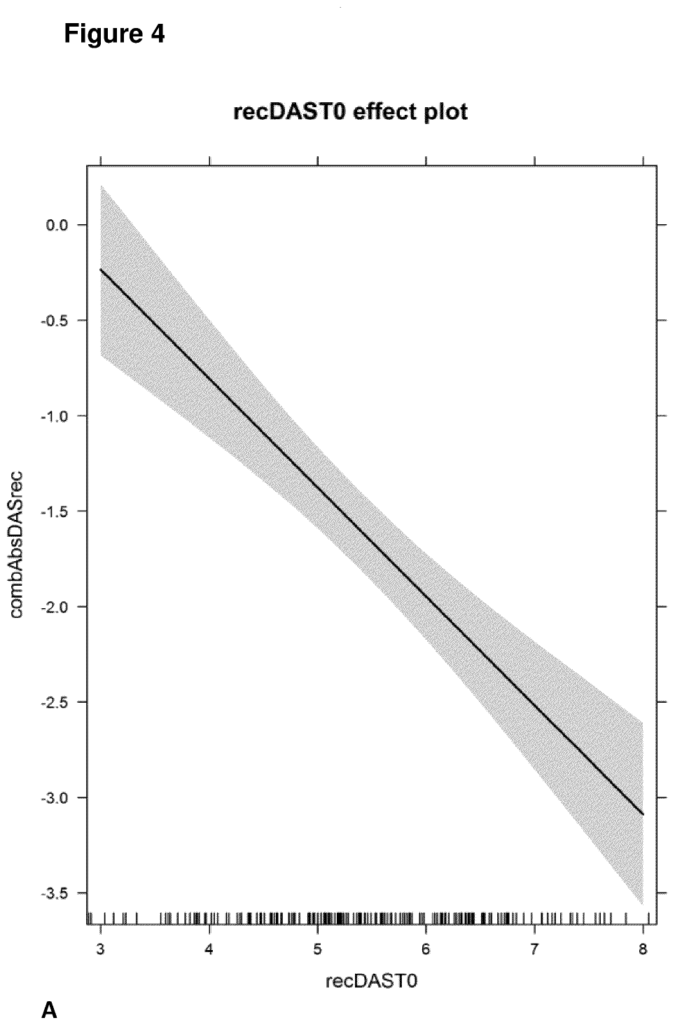

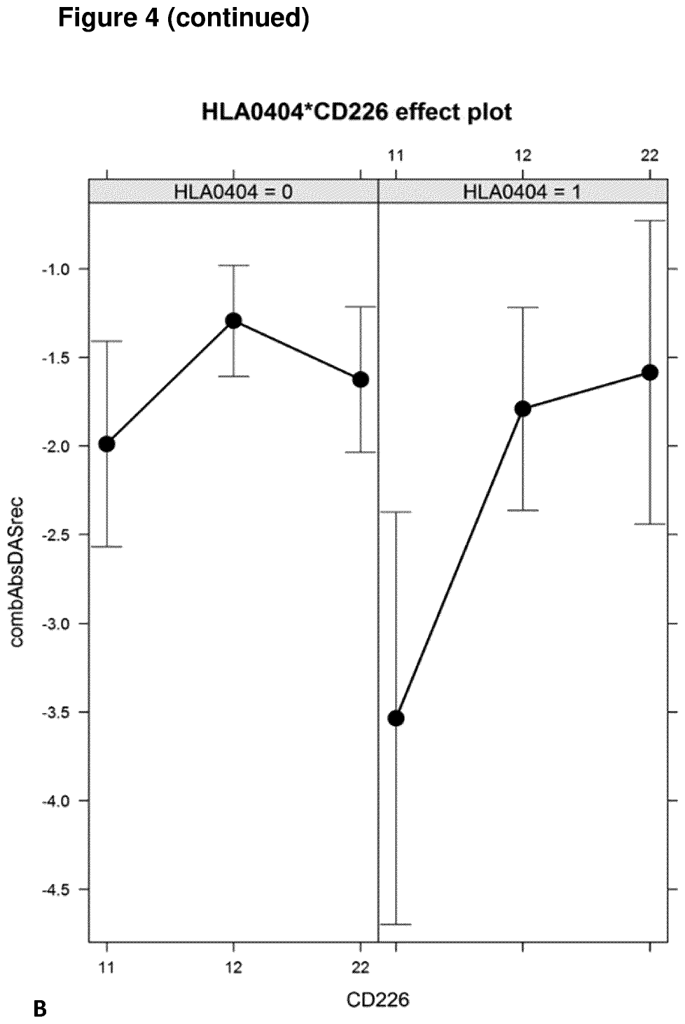

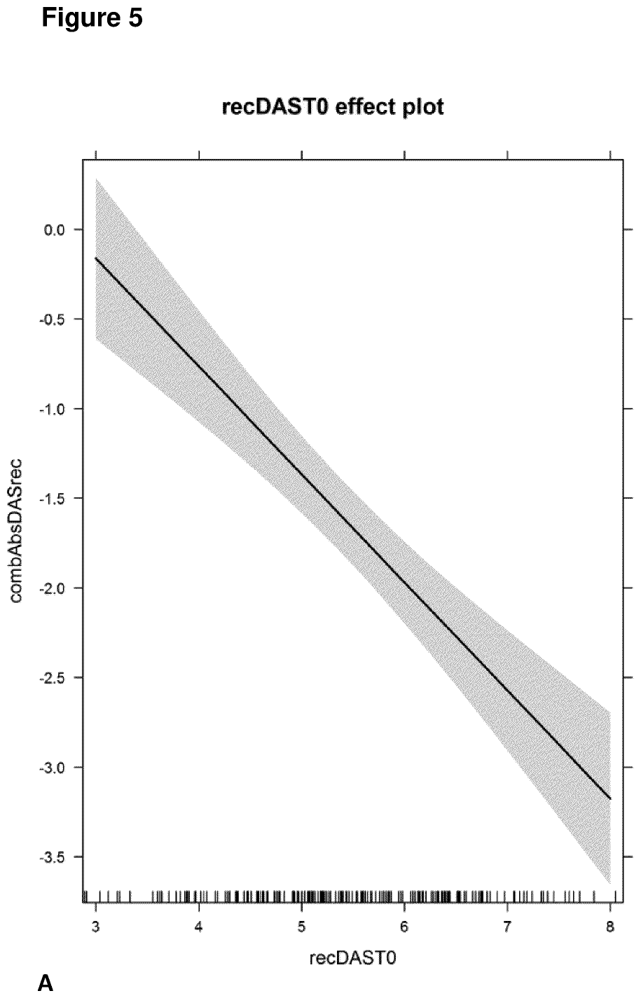

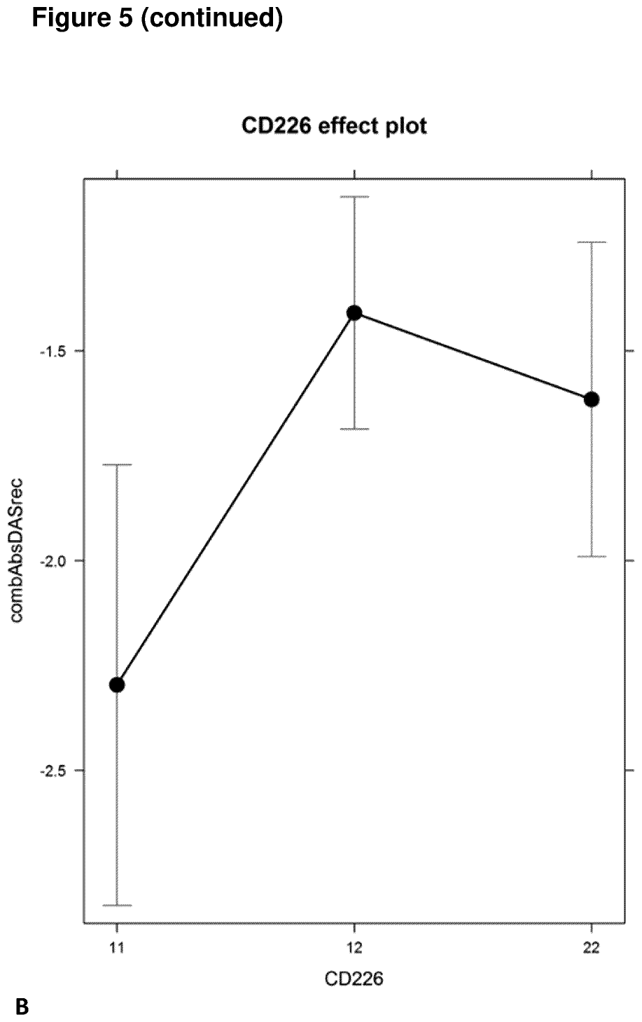

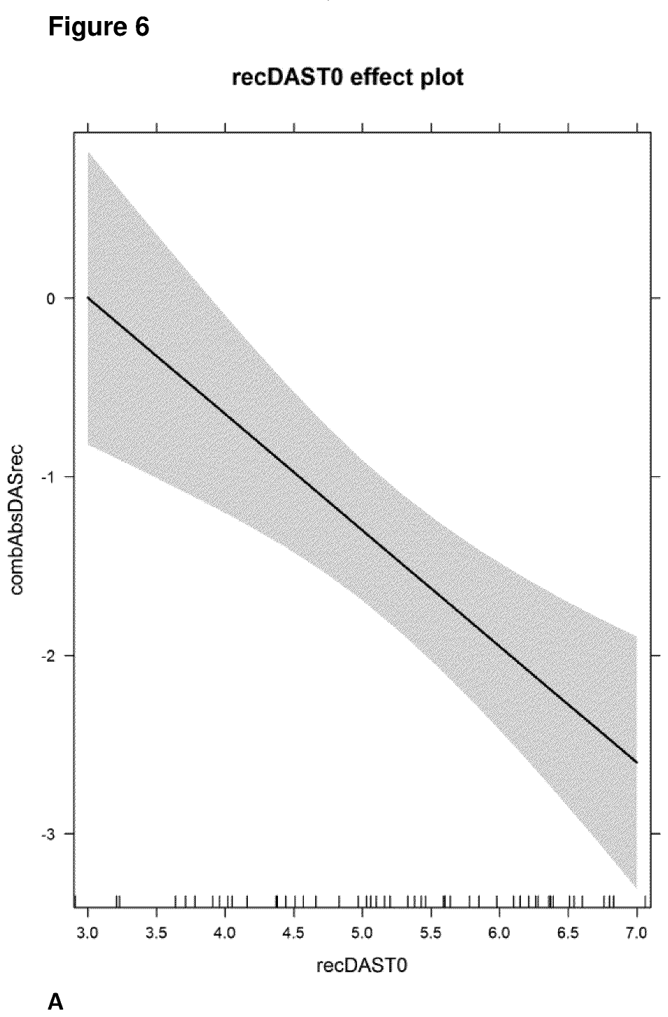

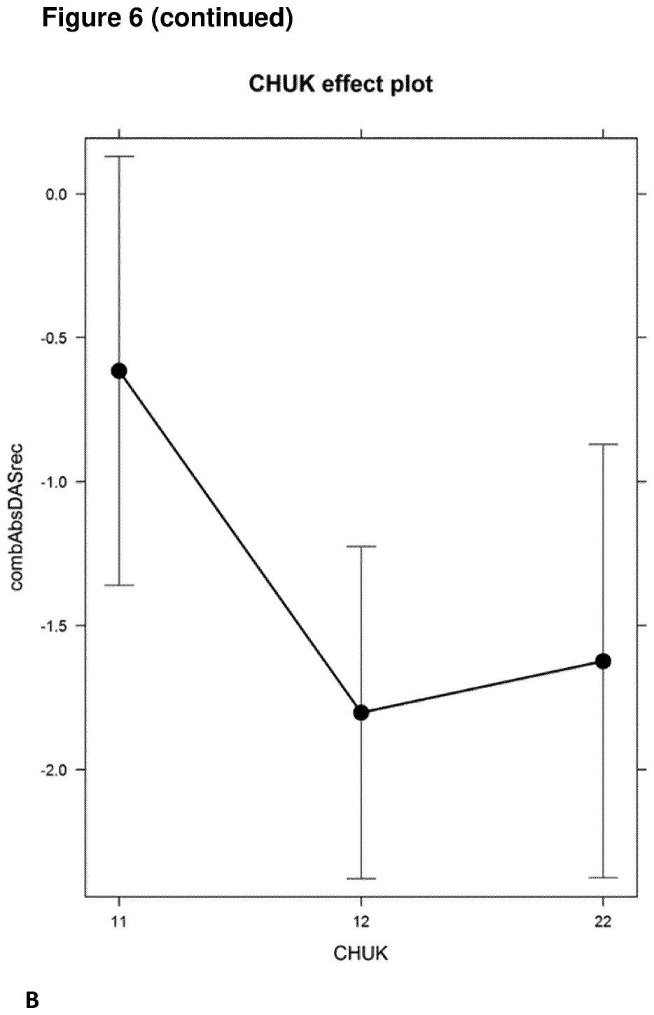

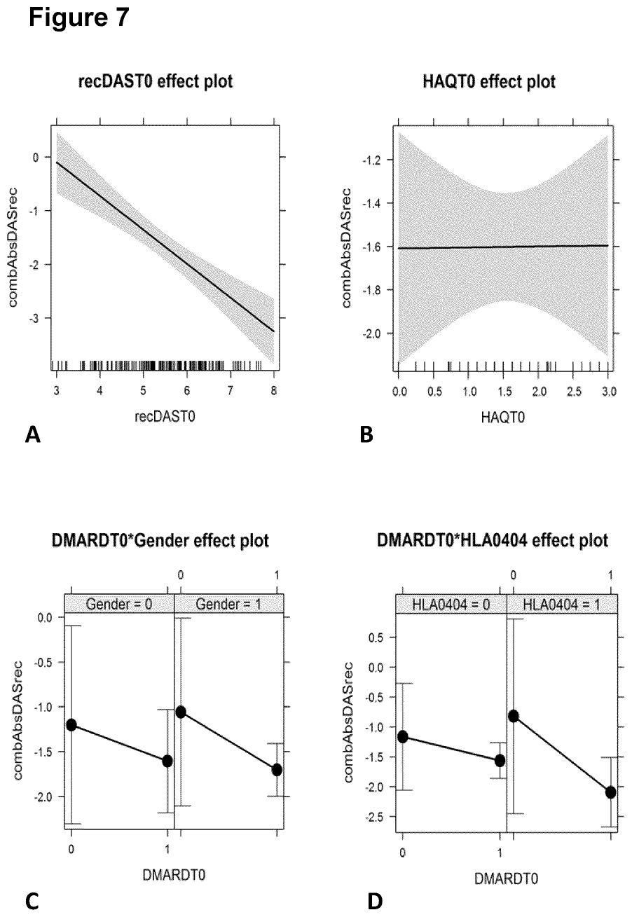

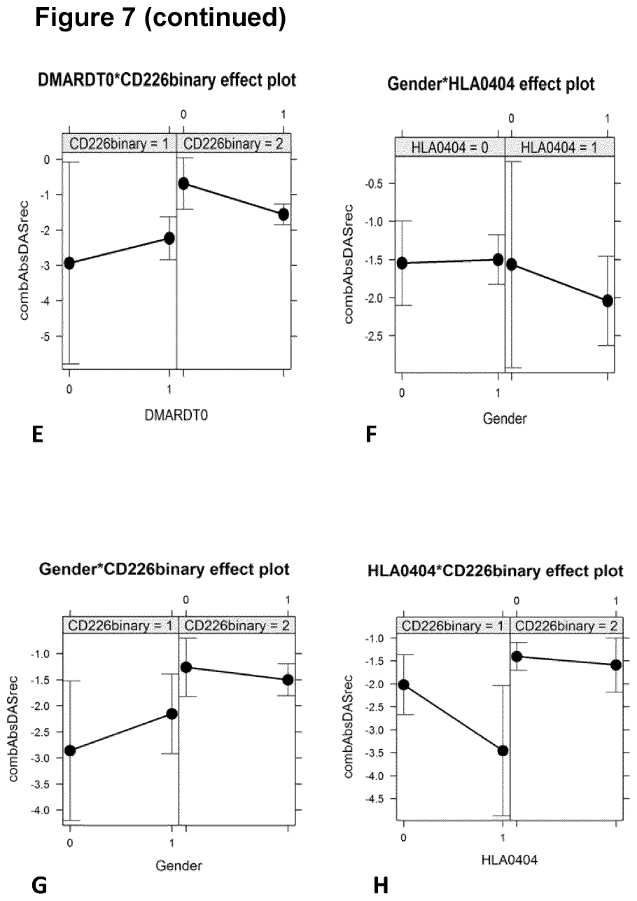

Baseline DAS28 score (p=1.39E-8) is highly significant, while DMARD (p=0.0507) is towards marginally significant at the conventional level (p=0.05). Subsequently, baseline DAS28 was built into a base model, with DMARD and all the genetic factors added individually to the base model to examine their association with ΔDAS28. Briefly, a series of linear regression models were fitted with baseline DAS28, plus one genetic factor or one other factor. The other factors considered here included rheumatoid factor (RF) status, anti-CCP status, the type of anti-TNF used and concurrent DMARD use. Screening through all the genetic factors and the other listed factors, only CD226 was found to contribute significantly (at the level of alpha=0.05) to ΔDAS28 after adjusting the effect of baseline DAS28 score (FIG. 3B). The HLA-DRB1*0404 haplotype, although found to be significantly associated with ΔDAS28 in the individual factor analysis above, is no longer significant after correcting for the effects of baseline DAS28 score.

In summary, baseline DAS28 score and CD226 together act as predictors of anti-TNF response which provide an adequate model in this cohort of RA patients. The results of this model are shown in Table 8, and the effects of baseline DAS28 and CD226 genotypes are depicted in FIG. 3. As can be seen from this figure, the homologous CD226 genotype 11 conforms to the best response, while patients with either genotype 12 or 22 have significantly worse response than genotype 11.

TABLE 8
The linear model with two predictors: baseline DAS28 and CD226

| Variable | Estimate | Std. Error | t value | Pr(>\|t\|) |
|---|---|---|---|---|
| (Intercept) | 0.957839 | 0.531244 | 1.803014 | 0.072946 |
| baseline | −0.60283 | 0.084762 | −7.11208 | 2.17E−11 |
| DAS28 | | | | |
| CD226 (12) | 0.886626 | 0.300816 | 2.947406 | 0.0036 |
| CD226 (22) | 0.680279 | 0.326803 | 2.081615 | 0.038697 |

After correcting for the effects of resDAST0, no other genetic factor or other factors appear to contribute significantly to the delta DAS28 score. Therefore the two predictors recDAST0 and CD226 together provide an optimal model to describe the response of this cohort of RA patients. Given the finding about HLA-DRB1*0404 in the individual factor analysis, we further investigated whether it might still have predictive value in the base model with the DAS28 recorded by the clinical team in patient records at baseline TO (recDAST0) and CD226 as built-in predictors. First, HLA-DRB1*0404 was added to this base model without interaction with CD226. In this simple interaction-free model, HLA-DRB1*0404's effects on 4DAS28 is just short of statistical significance (p=0.0506) at the conventional level (alpha=0.05). Secondly, we considered the interaction between CD226 and HLA-DRB1*0404 in the model, and found interesting results. There are significant differences among the 6 genotypes of CD226-HLA-DRB1*0404 combinations; Homozygous (11) absence of the CD226 rs763361 SNP and presence of HLA-DRB1*0404 allele represents the most responsive genotype, which is significantly better than most of other 5 combinations (FIG. 3C).

TABLE 9
Etanercept only-A linear model with two predictors:
baseline DAS and CHUK

| Variable | Estimate | Std. Error | t value | Pr(>\|t\|) |
|---|---|---|---|---|
| (Intercept) | 2.770363 | 0.903554 | 3.066074 | 0.003465 |
| recDAST0 | −0.65092 | 0.163906 | −3.97131 | 0.000225 |
| CHUK12 | −1.18807 | 0.46805 | −2.53833 | 0.014234 |
| CHUK22 | −1.0093 | 0.530834 | −1.90134 | 0.062914 |

Patients who received etanercept only: after correcting for effects of resDAST0, no other genetic factor or other factors appear to contribute significantly to the delta DAS28 score. Therefore the two predictors here, recDAST0 and CHUK together provide an optimal model to describe the response of RA patients who received etanercept only.

### Discussion

This study has tested for the first time individual allele associations with ADAS28 across a range of anti-TNF treatments. This is also the first study to report a combined predictive model which indicates that patients with presence of HLA-DRB1*0404 and absence of CD226 SNP rs763361 exhibit the largest reductions in DAS28 after anti-TNF treatment.

HLA-DRB1*0404 carriers have been historically associated with predisposal to a more severe arthritis phenotype and higher disease activity. The current study confirms that patients with the HLA-DRB1*0404 haplotype manifest significantly elevated baseline disease activity. Of particular interest in the current study, the presence of the HLA-DRB1*0404 allele was independently associated with a significantly higher drop in disease activity after anti-TNF treatment.

The presence of CD226 SNP rs763361 in the study population was associated with significantly reduced responses to anti-TNF treatment. The combined predictive model of the present invention helps control for the possibility that larger changes in ΔDAS28 are not solely due to higher baseline DAS28 in HLA-DRB1*0404 carriers. In the predictive model, only the CD226 SNP contributes to significant drops in disease activity post anti-TNF, once the potentially confounding effects of baseline DAS28 are corrected.

### Biological Significance

It is thought that anti-TNF targeting of inflammatory cells with membrane bound TNF enhances antibody dependent cellular cytotoxicity (ADCC) by macrophages and natural killer cells. Cell surface activating and inhibitory killer cell immunoglobulin-like receptors regulate natural killer cell functions via HLA class I molecule interaction. So it is reasonable to postulate that although the *0404 allele confers higher disease risk and activity, it may also positively modify ADCC mediated apoptosis and clearance by natural killer cells.

CD226 is involved in the effector functions of T helper cells and peripheral T cells exhibit increased CD226 expression in rheumatoid arthritis. The rs763361 SNP located in exon 7 of CD226 is a C/T polymorphism that confers a glycine 307-to-serine (Gly307Ser) change within the cytoplasmic tail of the CD226 receptor. This variant is strongly associated with susceptibility to multiple autoimmune conditions including type 1 diabetes, multiple sclerosis and RA. The biological consequence of the variant remains unclear, though it has been hypothesized that downstream effects on phosphorylation at Ser329 may be affected, which is required for cell activation via lymphocyte function-associated antigen 1 (LFA1).

### Clinical Significance

The ability to correctly predict responders for relatively high cost biologic treatments remains a lofty goal. Previously mentioned studies have reported a number of promising genotypes, but many observe that though associations may be strong or statistically significant, they may be of limited clinical benefit in managing patients. The ability of our model to correctly predict true responders (test sensitivity) is poor (23% HLA0404 and 19% CD226; Table 10). However, the ability to distinguish future responders with a positive HLA0404 test and a negative CD226 test was good with positive predictive values of 82% and 83%, respectively.

TABLE 10
Sensitivity and specificity of two potential predictors

| | Biomarkers | HLA-DRB1*0404 | CD226 rs763361 |
|---|---|---|---|
| | Population | n = 51 positive | n = 197 |
| | Test | (1); n = 187 | positive (12, 22), |
| | Values | negative (0) | n = 41 |
| | | | negative (11) |
| | Genotype | positive (1) | negative, |
| | association | associated with | wildtype (11) |
| | | greater ΔDAS28 | associated |
| | | | with greater |
| | | | ΔDAS28 |
| | Genotype | 21% | 17% |
| | prevalence | | |
| Responder | TP | 42 | 34 |
| | FN | 139 | 146 |
| Non- | TN | 52 | 54 |
| responder | FP | 9 | 7 |
| | Sensitivity | 23% | 19% |
| | TP/(TP + FN) | | |
| | Specificity | 85% | 89% |
| | TN/(TN + FP) | | |
| | NPV TN/ | 27% | 27% |
| | (FN + TN) | | |
| | PPV TP/ | 82% | 83% |
| | (TP + FP) | | |
| | FDR 1-PPV | 18% | 17% |

TP = true positive;
FN = false negative;
TN = true negative;
FP = false positive;
NPV = negative predictive value;
PPV = positive predictive value;
FDR = false discovery rate

This ‘future responder’ HLA0404-CD226 combined genotype represents 17% of the study population. Though not commercially viable alone, it may be increased if combined with other strongly associated genotypes and stratification of patients by significantly elevated baseline DAS28. The clinical validity and utility of a combined multi-parameter test would need to be assessed in an independent and much expanded cohort of patients.

### Other Observations

The study cohort was modest in size, but with adequate power to detect significant associations. Our power calculations indicate that a sample size of 150 patients can provide >80% power (at the conventional significance level alpha=0.05) to identify a genetic factor with a small to medium effect size (Cohen's f-squared=0.10) additional to our main model described above (baseline DAS28 plus CD226). As our dataset contains over 200 RA patients, therefore it provided adequate statistical power for the linear regression analysis conducted. It is also interesting to note that responders (moderate and good) appeared to have higher baseline tender joint counts (TJCs) compared to non-responders. This may suggest that the there is a different disease process associated with those that are responding versus those not responding. Further investigation of the biological pathways associated with TJC factors including the influence of the *0404 allele may be worthy of further study.

### Conclusions

Since anti-TNF treatments remain expensive, a pharmacogenetic approach to stratify patient populations could provide a reliable means to rationalise their use in those most likely to receive benefit.

All values are mean with standard deviation (s.d.), or percentage where indicated (%).

DAS28 refers to 28 joint disease activity score. DMARD refers to disease modifying anti-rheumatic drug. HAQ refers to health assessment questionnaire. CRP refers to C-reactive protein. SJC refers to swollen joint count. TJC refers to tender joint count.

HLA class II histocompatibility antigen, DRB1 beta chain is a protein that in humans is encoded by the HLA-DRB1 gene. HLA-DRB1*0404 is a shared epitope (SE) allele. cl REFERENCES

McGeough C M, Berrar D, Wright G, Mathews C, Gilmore P, et al. (2012) Killer immunoglobulin-like receptor and human leukocyte antigen-C genotypes in rheumatoid arthritis primary responders and non-responders to anti-TNF-alpha therapy. Rheumatol Int 32(6): 1647-1653.

Middleton D, Williams F, Halfpenny I A. (2005) KIR genes. Transpl Immunol14(3-4):135-42.

TABLE 11
Sequences

| SEQ ID NO: | Name | Sequence |
|---|---|---|
| 1 | HLA DRB1*0404 | cacgtttctt ggagcaggtt aaacatgagt |
| | | gtcatttctt caacgggacg gagcgggtgc |
| | | ggttcctgga cagatacttc tatcaccaag |
| | | aggagtacgt gcgcttcgac agcgacgtgg |
| | | gggagtaccg ggcggtgacg gagctggggc |
| | | ggcctgatgg cgagtactgg aacagccaga |
| | | aggacctcct ggagcagagg cgggccgcgg |
| | | tggacaccta ctgcagacac aactacgggg |
| | | ttgtggagag cttcacagtg cagcgg |
| 2 | CD226 | atgagtacat aagagtcatt actaatgcac |
| | (sequence 69864267- | tcatgtcaag aataagctta aactctagtc |
| | 69864536 shown, SNP at | tttggtctgc gagagaaggt tggatagttg |
| | position 69864406) | acataaatat cctctcttgt atcatccatg |
| | | gattgattgg taggttgact ggtagagatg |
| | | ggacttctat agttattggg tgcctagaaa |
| | | gacaaacagc agagagtgtc aataattcac |
| | | tgcatttcaa caactaatga agacattcaa |
| | | aacatacctc tcataaatgc aggcatgata |
| 3 | F primer for HLA DRB1*0404 | cgtttcttgg agcaggttaa aca |
| 4 | R primer for HLA DRB1*0404 | ctcgccgctg cactgtg |
| 5 | F primer for rs763361 | tttggtctgc gagagaaggt |
| 6 | R primer for rs763361 | tgcctgcatt tatgagaggt |

1. A method for predicting responsiveness to anti-tumour necrosis factor therapy of rheumatoid arthritis in a subject, the method comprising the steps of:
a) Providing a biological sample;
b) detecting the presence, absence, or quantitative level of a first marker or an expression product thereof, wherein the first marker is at the HLA-DRB1 gene;
c) detecting the presence, absence, or quantitative level of a second marker, or an expression product thereof, wherein the second marker is at the CD226 gene;
d) correlating the presence, absence, or quantitative level of the first marker and the presence, absence, or quantitative level of the second marker; to the predicted responsiveness to anti-tumour necrosis factor therapy of rheumatoid arthritis in the subject.

2. The method of claim 1, wherein the first marker comprises at least part of a nucleic acid sequence selected from the nucleic acid sequence of the HLA-DRB1*0404 allele, and the nucleic acid sequence of SEQ ID NO: 1.

3. The method of any preceding claim, wherein the first marker comprises a nucleic acid sequence selected from the nucleic acid sequence of the HLA-DRB1*0404 allele, and the nucleic acid sequence of SEQ ID NO: 1.

4. The method of any preceding claim, wherein the second marker comprises a nucleic acid sequence selected from the nucleic acid sequence of the rs763361 cytosine single nucleotide polymorphism, and the nucleic acid sequence of SEQ ID NO: 2.

5. The method of any preceding claim, wherein absence of the second marker is homozygous absence of the second marker.

6. The method of any preceding claim, wherein the presence of the first marker indicates that the subject is predicted to be responsive to anti- tumour necrosis factor therapy of rheumatoid arthritis.

7. The method of any preceding claim, wherein the absence of the second marker indicates that the subject is predicted to be responsive to anti-tumour necrosis factor therapy of rheumatoid arthritis.

8. The method of any preceding claim, wherein the method further comprises the step of evaluating the DAS28 score of the subject.

9. The method of any preceding claim, wherein the method comprises a polymerase chain reaction method.

10. The method of any preceding claim, wherein the method comprises a biochip array method.

11. The method of any preceding claim, wherein detecting step (b) comprises: contacting the sample with at least one primer having a nucleic acid sequence defined by at least one of: SEQ ID NO: 3, 4, and the reverse complement each thereof; under conditions suitable for a polymerase chain reaction.

12. The method of any preceding claim, wherein detecting step (c) comprises: contacting the sample with at least one primer having a nucleic acid sequence defined by at least one of: SEQ ID NO: 5, 6, and the reverse complement each thereof; under conditions suitable for a polymerase chain reaction.

13. The method of any preceding claim, wherein the biological sample substantially comprises a blood sample.

14. An anti-tumour necrosis factor rheumatoid arthritis therapy response prediction kit, comprising at least one primer or probe having a nucleic acid sequence defined by any of SEQ ID NO: 3, 4, 5, and 6.

15. The kit of claim 14 further comprising a solid support, optionally further comprising instructions for use.

16. An in vitro method for predicting responsiveness to anti-tumour necrosis factor therapy of rheumatoid arthritis in a subject comprising the method of any of claims 1 to 13, wherein the presence of the first marker and the absence of the second marker indicates that the subject is predicted to be responsive to anti-tumour necrosis factor therapy of rheumatoid arthritis.


SEQUENCE LISTING (6 sequences)

SEQ ID NO: 1 - DNA, 266 bp, Homo sapiens
cacgtttcttggagcaggttaaacatgagtgtcatttcttcaacgggacggagcgggtgcggttcctggacagatacttctatcaccaagaggagtacgtgcgcttcgacagcgacgtgggggagtaccgggcggtgacggagctggggcggcctgatggcgagtactggaacagccagaaggacctcctggagcagaggcgggccgcggtggacacctactgcagacacaactacggggttgtggagagcttcacagtgcagcgg

SEQ ID NO: 2 - DNA, 270 bp, Homo sapiens
atgagtacataagagtcattactaatgcactcatgtcaagaataagcttaaactctagtctttggtctgcgagagaaggttggatagttgacataaatatcctctcttgtatcatccatggattgattggtaggttgactggtagagatgggacttctatagttattgggtgcctagaaagacaaacagcagagagtgtcaataattcactgcatttcaacaactaatgaagacattcaaaacatacctctcataaatgcaggcatgata

SEQ ID NO: 3 - DNA, 23 bp, Unknown
  synthetic oligo
cgtttcttggagcaggttaaaca

SEQ ID NO: 4 - DNA, 17 bp, Unknown
  synthetic oligo
ctcgccgctgcactgtg

SEQ ID NO: 5 - DNA, 20 bp, Unknown
  synthetic oligo
tttggtctgcgagagaaggt

SEQ ID NO: 6 - DNA, 20 bp, Unknown
  synthetic oligo
tgcctgcatttatgagaggt